(19) United States
(12) Patent Application Publication
(10) Pub. No.: US 2007/0185477 A1
(43) Pub. Date: Aug. 9, 2007

(54) Removable Cryogenic Probe Appliance

(76) Inventor: Michael D. Hooven, Cincinnati, OH (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 11/672,328

(22) Filed: Feb. 7, 2007

Related U.S. Application Data
(60) Provisional application No. 60/765,910, filed on Feb. 7, 2006.

(51) Int. Cl. A61B 18/02

(52) U.S. Cl. 606/23

(57) ABSTRACT

A removable cryogenic probe appliance is disclosed comprising a proximal portion and a distal portion, the proximal portion being releasably attachable to a cryogenic probe. The appliance further comprises a material having high thermal conductivity for conducting heat between said proximal and distal portions. A cryogenic probe system and methods of forming and using a cryogenic probe of selected shape are also disclosed, comprising providing a plurality of removable cryogenic probe appliances of differing shapes, selecting one of said appliances of the desired shape, and attaching the proximal portion of such selected appliance to a cryogenic probe.

### RELATED APPLICATIONS

This application claims the benefit of U.S. Provisional Application Ser. No. 60/765,910, filed Feb. 7, 2006 which is incorporated in its entirety by reference herein.

### FIELD OF THE INVENTION

The present invention relates to an apparatus and method for the ablation of human tissue, and more particularly, for ablation of cardiac tissue using a cryogenic probe.

### BACKGROUND OF THE INVENTION

Surgical ablation can be used to treat various medical conditions in the human body. One type of ablation employs freezing of the target tissue by a cryogenic probe. As used herein, “cryoablation” means the ablation of human tissue by a cryogenic probe.

Typically, cryoablation requires that a supply of cryogenic cooling fluid in the form of a liquid or gas circulate under pressure within a distal effector or appliance, referred to as a cryogenic probe. High pressure, low temperature liquefied gas is circulated through the probe to cool it to an extremely low temperature. When the probe is contacted with selected tissue to be ablated, heat is removed from the selected tissue until the temperature of the selected tissue falls at or below about 0° C. At about that temperature, the cells undergo irreversible electrical effects and are destroyed, or ablated.

Ablation of cardiac tissue to form one or more lines of ablated tissue comparable to the scar tissue formed in the so-called MAZE procedure has been found to be useful for the treatment of cardiac disorders, such as cardiac arrhythmias. Lines of ablation in the cardiac tissue create scar tissue that interrupts the path of errant electrical impulses in the heart tissue.

Such ablation may range from the ablation of a limited area of heart tissue such as around each pair of pulmonary veins, to a more complex series of ablations forming a strategic placement of lines to mimic the MAZE procedure to stop the conduction and formation of errant impulses. Ablation of cardiac tissue may be carried out in an open surgical procedure, where the breastbone is divided and the surgeon has direct access to the heart, or through a minimally invasive route.

As noted above, cryoablation of cardiac tissue is typically performed with a slender medical implement referred to as a probe. Due to the strength required to withstand circulating super-cooled liquefied gas, conventional probes are rigid and are pre-formed into a specific shape. To change the shape requires replacing the entire probe. The limited number of different shapes is also not conducive to use of such a probe on tissue having a shape different from that of the selected probe. Therefore, the user of a conventional rigid probe may have difficulty accessing the selected tissue to be ablated because the shape of the probe does not conform to the tissue. As a result, the user may not be able to create a precise line or region of ablation because of the difficulty of reaching certain areas of tissue with a rigid probe that does not conform to the shape of the tissue. This may lead to healthy tissue surrounding the ablation site being inadvertently damaged and/or ablated or incomplete formation of the desired ablation, allowing errant electrical signals to pass instead of being blocked.

Reusable cryogenic probes must be designed to withstand abuse over many years and multiple procedures. Reusable cryogenic probes are not malleable because of the danger of multiple uses and bendings causing a failure of the probe. Bending of the probe over time and multiple procedures could cause a weak point, and a subsequent leak in the probe. Any leak in a cryogenic probe tip could be catastrophic during surgery.

Consequently, there is a need for a cryogenic probe that is more convenient to use and/or more readily accommodates tissue of varying shape or size.

### BRIEF DESCRIPTION OF THE DRAWINGS

FIG. 1a is a side cross-sectional view of a disc-shaped cryogenic probe appliance of the present invention removably attached to a cryogenic probe.

FIG. 1b is a side cross-sectional view of another embodiment of a removable cryogenic probe appliance embodied in the present invention, removably attached to a cryogenic probe.

FIG. 2 is a side cross-sectional view of a removable cryogenic probe appliance of the present invention removably attached to a cryogenic probe by a friction fit.

FIG. 3 is a side cross-sectional view of a removable cryogenic probe appliance of the present invention removably attached to a cryogenic probe by a detent mechanism.

FIG. 4a is a perspective view of a removable cryogenic probe appliance of the present invention adapted to be removably mounted to a cryogenic probe by a bayonet lock.

FIG. 4b is a perspective view of a removable cryogenic probe appliance of the present invention removably attached to a cryogenic probe by a bayonet lock.

FIG. 5a is a perspective view of the removable cryogenic probe appliance of the present invention adapted to receive a probe with a snap, or socket attachment means.

FIG. 5b is a perspective view of the removable cryogenic probe appliance of the present invention removably attached to a probe by a snap or socket mechanism.

FIG. 6 is a side cross-sectional view of the removable cryogenic probe appliance of the present invention threadedly attached to a probe.

FIG. 7 is a side cross-sectional view of the removable cryogenic probe appliance of the present invention showing the appliance in a downwardly bent configuration.

FIG. 8 is a side cross-sectional view of the removable cryogenic probe appliance of the present invention showing the appliance in an upwardly bent configuration.

FIG. 9 is a side cross-sectional view of the removable cryogenic probe appliance of the present invention showing the appliance in a straightened configuration.

FIG. 10 is a side cross-sectional view of the removable cryogenic probe appliance of the present invention showing the appliance in a straight and downwardly facing configuration.

FIG. 11 is a side cross-sectional view of a disc-shaped embodiment of the removable cryogenic probe appliance of the present invention in a downwardly facing configuration.

FIG. 12 is a perspective view of another embodiment of a removable cryogenic probe appliance embodied in the present invention, removably attached to a cryogenic probe.

FIG. 13 is a perspective view of the embodiment shown in FIG. 12, showing the proximal portion of the appliance extending along the length of the distal portion of a cryogenic probe at the area of attachment between the two portions.

FIG. 14 is a cross-sectional view of another embodiment of the cryogenic probe appliance of the present invention removably attached to a cryogenic probe.

FIG. 15 is a perspective view showing the malleability of a cryogenic probe appliance.

### SUMMARY OF THE INVENTION

In accordance with one aspect of the present invention, a removable cryogenic probe appliance is provided which includes a proximal portion and a distal portion and comprises a material having high thermal conductivity. The proximal portion is releasably attachable to a cryogenic probe. This allows for increased convenience in that a plurality of such appliances of differing shapes may be provided and readily attached to or removed from a cryogenic probe as circumstances require, without the need for disassembling or replacing the cryogenic probe itself.

When the removable cryogenic probe appliance is attached to the cryogenic probe, there preferably is a sufficiently large area of contact between the proximal portion of the appliance and the distal portion of the probe at the point of attachment, such that efficient heat transfer can occur between the probe and the appliance. To further achieve efficient heat transfer, the probe appliance material preferably has a thermal conductivity of at least about 100 W·m⁻¹·K⁻¹ and comprises material such as aluminum or copper. This allows the attached appliance to maintain substantially the same temperature as the probe during use.

In accordance with another aspect of the present invention, a cryogenic probe appliance may be provided for removable attachment to a cryogenic probe, wherein at least a portion of the appliance is malleable and may be formed or bent into a different desired shape for treating a particular tissue area. The different shapes of cryogenic appliances that are provided may include a shape suitable to form a straight ablation line, a curved ablation line, or a larger two dimensional region of ablation. Such an appliance provides substantial advantages to cryogenic procedures—allowing essentially custom shaping of the operative end of the probe and avoiding the need for removing and replacing the probe itself or the need for multiple probes of various shapes.

A further object of the present invention is to combine a disposable malleable tip with a reusable rigid probe, eliminating the danger of multiple reuses of a malleable probe

### DETAILED DESCRIPTION OF THE INVENTION

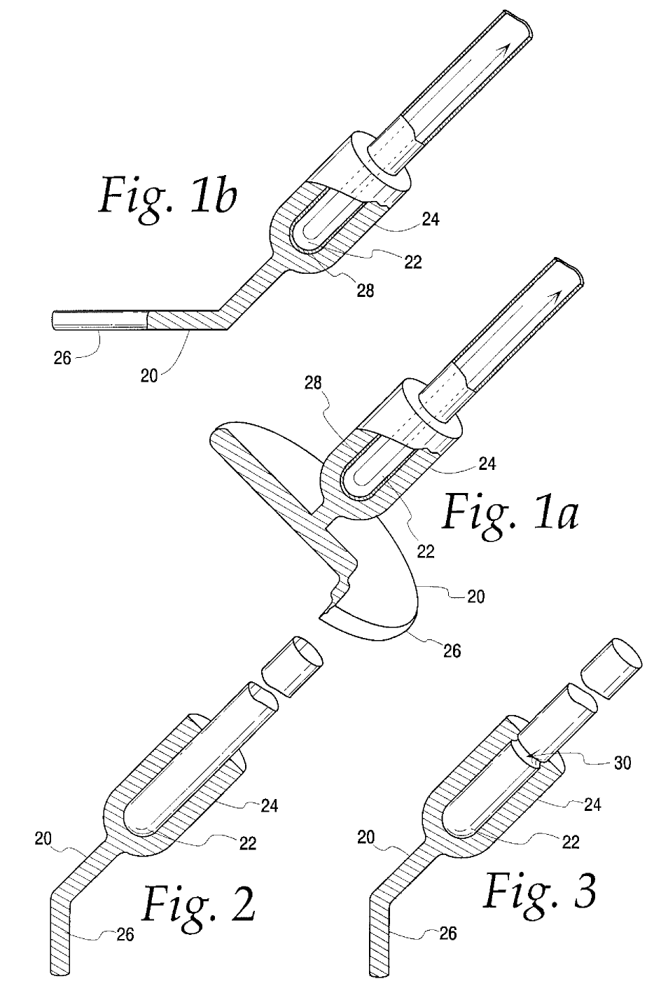

Turning now to a more detailed description, FIGS. 1a and 1b generally illustrate embodiments of a removable cryogenic probe appliance 20 removably attached to the distal portion or end of a cryogenic probe 22. The probe appliance 20, which acts as an extension of a cryogenic probe 22, provides substantial advantages over the typical cryogenic probes and cryogenic ablation procedures.

As shown in FIGS. 1a and 1b, the cryogenic probe appliance 20 includes a proximal portion, generally at 24, and a distal portion, generally at 26. The proximal portion, as illustrated, is adapted to removably receive the distal end of the cryogenic probe 22.

As illustrated, the cryogenic probe 22 comprises an elongated cylinder shaft terminating in a rounded tip and including passageways for passing liquefied gas through the probe. The probe is typically made of stainless steel or other high strength material to withstand the pressure of liquefied gas therewithin. For removable attachment to the cryogenic probe 22, the probe appliance of the present invention preferably has a receiving cavity or bore 28 located in the proximal portion 24 for receiving the end of the probe. The bore 28 is sized to approximate the size of the probe and receive the probe so that the surfaces of the bore and probe are in close contact or with only very slight spacing between them to enhance heat transfer between the probe 22 and the probe appliance 20. A heat transfer agent, such as a liquid or gel, may also be employed on the surface of the probe or bore to fill any gap between the surfaces and better enable heat transfer between them.

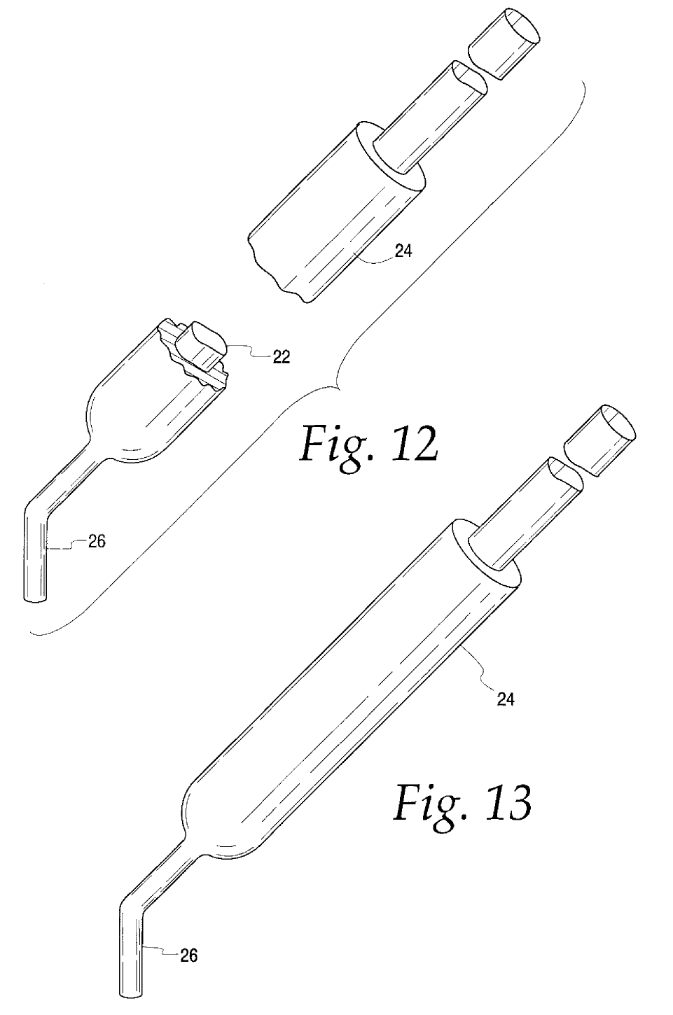

As further illustrated in FIGS. 12 and 13, the proximal portion of the appliance preferably is sized to receive a sufficient length of the distal portion of the cryogenic probe to provide for substantial surface-to-surface contact to enhance transfer of heat between the contacting areas of the two portions.

The distal portion or end 26 of the cryogenic appliance 20 may take any desired shape as needed for the particular tissue to be ablated. In FIG. 1a, for example, the distal portion of the appliance is disc-shaped for ablating a large, two-dimensional region of tissue. In contrast, the distal portion of the appliance in FIG. 1b is an elongated member angled to form a straight distal portion that may be useful in forming a line of ablation on tissue. When forming a line of ablation, it is preferable to have a narrow area of surface contact between tissue and probe appliance. This can be achieved by minimizing the amount of material that comprises the distal portion of the appliance that is not in contact with selected tissue. This allows for a relatively quick transfer of heat from the appliance to the tissue. This further allows for a precise area of selected tissue to be cooled to a temperature low enough and to maintain the tissue at such temperature to ablate the tissue substantially entirely through its thickness, i.e., transmurally. When the area of surface contact between the tissue and probe appliance is sufficiently limited or narrow, the time for ablation preferably does not exceed a few minutes. Other shapes of the probe appliance may be provided that are particularly suited for different procedures or different organs or tissue to be ablated.

To provide for efficient transfer of heat between the proximal and distal portions 24 and 26 of the appliance 20, the appliance preferably comprises a material of high thermal conductivity. The appliance may be entirely made of such material or may be of laminated or other construction where materials of differing thermal conductivity are used, or certain surfaces may be insulated to avoid injury to tissues that are not to be ablated. For purposes of this description, high thermal conductivity is preferably about 100 W·m⁻¹·K⁻¹ or greater and more preferably about 200 W·m⁻¹·K¹ or greater, and still more preferably about 300 W·m⁻¹·K⁻¹ or greater. Potential highly thermally conductive materials useful in the present invention include copper (which may be gold plated), with a thermal conductivity of about 386 W·m⁻¹·K⁻¹, aluminum, with a thermal conductivity of about 237 W·m⁻¹·K⁻¹, silver or silver alloy, with a thermal conductivity of about 429 W·m⁻¹·K⁻¹, or brass, with a thermal conductivity of about 120 W·m⁻¹·K⁻¹. Copper or gold plated copper may be the preferred material, but the invention is not limited to the particular material.

The mass of the high thermal conductivity material may be varied as desired for the individual applications. It may be desirable or preferred in some applications to provide substantial or maximum mass of high thermal conductive material to allow the appliance to act as a heat sink upon contact with tissue. In other applications, it may be desirable to have reduced or minimum mass in the appliance to enhance the rate of temperature change of the appliance.

As noted above, to enhance heat transfer between the probe and probe appliance, substantial surface-to-surface contact should be maintained between the probe and the appliance at the point of attachment such that heat transfer can take place between the probe and the attached appliance. Therefore, the proximal portion of the appliance should be sized and shaped to achieve a substantial and preferably maximum practical surface area of contact with the distal portion of the probe to allow for heat to be transferred readily between the two portions. More specifically, the proximal portion of the appliance may be of an extended length to receive a substantial extent of the distal end of the probe.

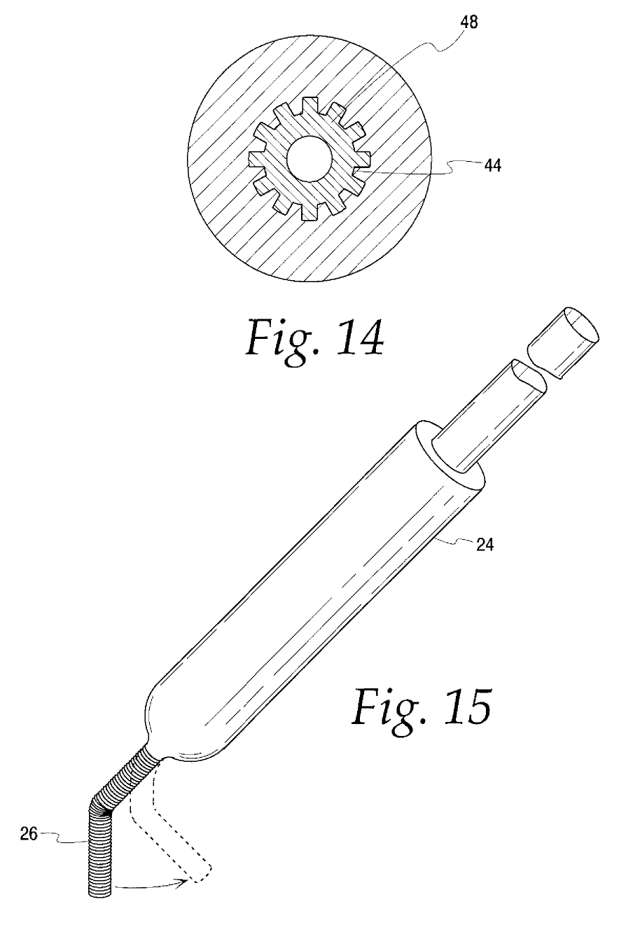

Another approach to increasing the surface area of contact between the probe and appliance is illustrated in FIG. 14. To increase surface-to-surface contact, as seen in FIG. 14, the interior of the proximal portion of the removable appliance may be configured with radially extending ribs or fins 44, which inter-fit with radial grooves 48 of the cryogenic probe, or vice versa. Other shapes and configurations such as threads or other arrangements may also be employed to increase surface-to-surface contact to provide for increased rate of heat transfer between the probe and the appliance so that the appliance reaches and maintains substantially the same temperature as the cryogenic probe to which it is attached during ablation.

The cryogenic probe appliance of the present invention can have any of a variety of preformed shapes. With several different shapes for different types of ablation or different tissue types, the desired shape can be selected and the appliance attached to the probe. If a different shape is desired later, the first appliance can be easily removed and the appliance with the desired shape attached to the probe. This avoids the need of removing or replacing the probe itself and provides an array of useful probe appliances of different shapes less expensively than a similar array of differently shaped cryogenic probes.

In accordance with another aspect of the present invention, the probe appliance may be malleable, in whole or in part, as illustrated in FIG. 15, which depicts a removable appliance with a corrugated distal portion permitting bending to form a desired shape. Preferably, in this aspect, at least a portion of the appliance is sufficiently malleable to allow the user to shape it manually, such as by bending or twisting, to form it into a desired shape. This allows the user to create unique custom shapes for particular surgical needs that may not be met by a probe of pre-formed shape. For example, the distal portion of a straight probe appliance may be malleable to allow it to be bent into a curve or a combination of curves and straight sections to reach or contact a particular tissue surface. The appliance may be malleable as a result of material of the appliance and/or as a result of design features, such as the corrugated design shown in FIG. 15.

As shown in the attached drawings, the probe appliance of the present invention is not limited to a particular means of attachment to the probe. A variety of mechanisms can be used for easy and secure attachment and removal. FIG. 2 shows the probe appliance 20 removably attached to a probe 22 by a friction fit. A friction fit is achieved with an appliance having a proximal portion with a narrower internal diameter, such as a slightly tapered diameter, than the outer diameter of the distal end of the probe. When the appliance and probe are pushed together, the appliance slides up and around the distal portion of the probe, thus forming a tight fit. Frictional forces hold the appliance securely in place around the distal portion of the probe during use. However, when use of the apparatus has been completed, the appliance can be removed from the probe by pulling the probe appliance and probe away from each other until the appliance slides off the end of the probe.

FIG. 3 shows the probe appliance 20 removably attached to a cryogenic probe 22 by a detent mechanism. A cryogenic probe having a circumferential depression 30 around the exterior of the distal portion of the probe is shaped to receive a circumferential lip or spring-loaded ball located on the interior of the proximal portion of the appliance. When the appliance and probe are pushed together, the lip engages the depression, thus attaching the two portions together. After use, the appliance can be removed from the probe by simply pulling the two portions apart until the lip and depression become disengaged.

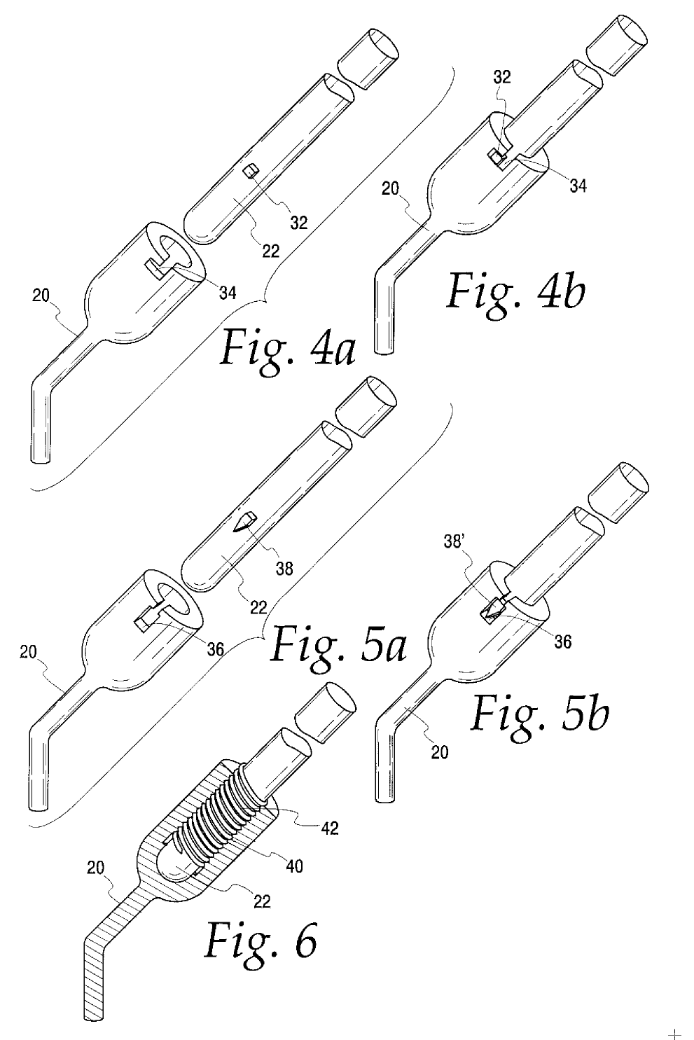

FIGS. 4a and 4b illustrate the removable attachment of the cryogenic probe appliance 20 to a cryogenic probe 22 by a bayonet lock. In FIG. 4a, a probe is spaced apart from an appliance, wherein the probe comprises at least one projecting pin or member 32. The pin is removably receivable by a J-shaped slot 34 in a corresponding probe appliance. When the probe and appliance are pushed together, and relatively rotated in opposite directions, the pin is secured in the slot. After use, the probe and appliance can once again be rotated in opposite directions and pulled apart to disengage the pin from the slot, and remove the appliance from the distal portion of the probe.

As seen in FIGS. 5a and 5b, the probe appliance 20 can be removably attached to a probe 22 by a snap mechanism. In FIG. 5a, the appliance is shown spaced apart from a cryogenic probe. The proximal portion of the appliance has a socket or cavity 36 to removably receive a probe having a protrusion 38 on its distal end. The probe and appliance are coupled together, as shown in FIG. 5b, when the two portions are pushed together, forcing the protrusion into socket or cavity 36. Once again, the appliance can be removed from the probe when the two portions are pulled apart, until the protrusion is disengaged from the socket.

FIG. 6 shows a cryogenic probe 22 and probe appliance 20 threadedly attached. The interior of the proximal portion of the appliance comprises grooves 40 shaped to engage corresponding threads 42 on the exterior of the distal portion of a probe. The two portions can be rotated about their axes to screw together.

The location of parts on the embodiments described above may be reversed or modified according to design choice. Also, other modes or manners of attachment of the probe appliance 20 and probe 22 may be used without departing from the present invention.

As described briefly above, the cryoablation of cardiac tissue may require any number of differently-shaped probes to complete the desired ablation or the creation of the desired pattern of ablation lines. Therefore, the removable cryogenic probe appliance provides a number of advantages. First, either a plurality of pre-formed probe appliances of different shapes may be provided or at least a portion of the appliance may be malleable so that it can be shaped into a desired configuration. As noted above, a malleable probe appliance allows the user to shape the appliance to conform to the selected tissue to be ablated. By conforming the distal portion of the appliance to selected tissue, better ablation of the selected tissue can be achieved, and damage to healthy tissue can be avoided.

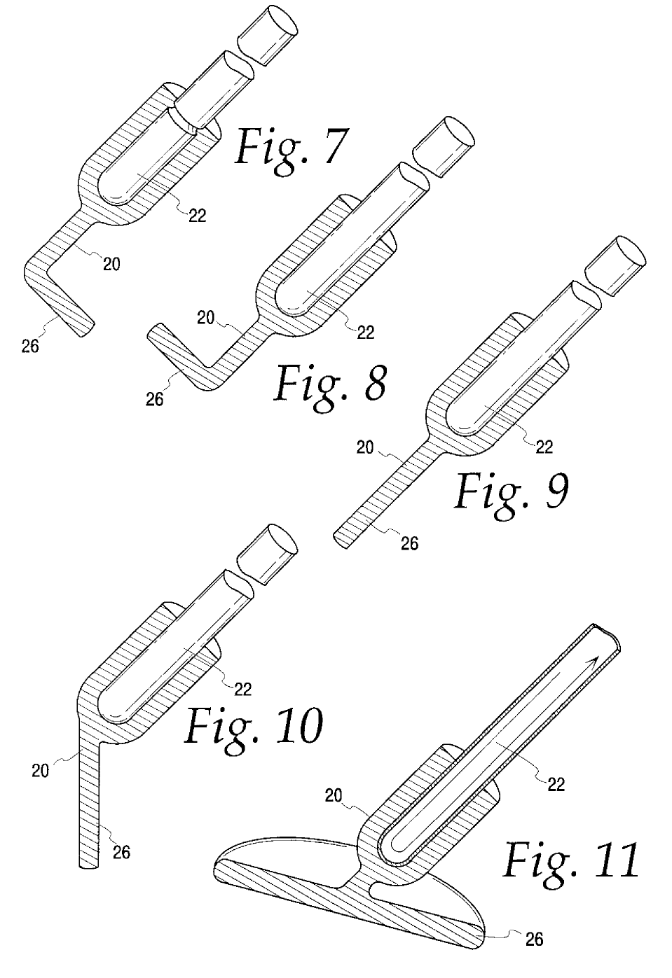

FIGS. 7-11 illustrate various embodiments that can be employed when the distal portion of the removable cryogenic probe appliance is pre-formed or shaped into a desired configuration. For example, in FIG. 7, the probe appliance 20 is long and narrow and terminates at a 90° angle. This shape may be useful for forming short straight ablation lines. FIG. 8 is similar, but with the distal end angled in a different direction. FIG. 9 shows a probe appliance 20 with a straight distal end that may be useful for ablating point sources or for contacting and dragging along tissue to be ablated. FIG. 10 is similar to FIG. 9, but with the entire distal portion of the probe appliance 20 extending at an angle.

While the probe appliance of the present invention is useful in forming lines of ablation, it is not limited to such applications. FIG. 11 shows a probe appliance 20 of the present invention for forming larger two-dimensional areas of ablation.

While the invention has been described in terms of certain preferred embodiments, it is not limited to the precise embodiments shown or to the particular features, shapes or sizes illustrated. A variety of changes may be made without departing from the present invention as defined by the appended claims.

What is claimed is:

1. A removable cryogenic probe appliance comprising
a proximal portion and a distal portion, the proximal portion being releasably attachable to a cryogenic probe;
the appliance further comprising a material having high thermal conductivity for conducting heat between said proximal and distal portions.

2. The removable cryogenic probe appliance of claim 1 wherein at least the distal portion is flexible.

3. The removable cryogenic probe appliance of claim 1 wherein the material has a thermal conductivity of greater than about 100 W·m⁻¹·K⁻¹.

4. The removable cryogenic probe appliance of claim 1 wherein the material comprises aluminum.

5. The removable cryogenic probe appliance of claim 1 wherein the material comprises copper.

6. The removable cryogenic probe appliance of claim 1 wherein the material is malleable.

7. The removable cryogenic probe appliance of claim 1 wherein the appliance is shaped to form a desired pattern of ablation in human tissue.

8. The removable cryogenic probe appliance of claim 1 wherein the appliance is adapted to contact cardiac tissue.

9. The removable cryogenic probe appliance of claim 1 wherein the appliance is shaped to form a line of ablation.

10. The removable cryogenic probe appliance of claim 1 wherein the appliance is shaped to form a two dimensional region of ablation.

11. The removable cryogenic probe appliance of claim 1 wherein the proximal portion includes means for releasably attaching to a cryogenic probe.

12. The removable cryogenic probe appliance of claim 1 wherein the proximal portion includes threads for releasable attachment to a cryogenic probe.

13. The removable cryogenic probe appliance of claim 1 wherein the appliance is frictionally engageable to a probe.

14. The removable cryogenic probe appliance of claim 1 wherein the proximal portion includes a bayonet lock for attaching to a probe.

15. The removable cryogenic probe appliance of claim 1 wherein the proximal portion includes a detent mechanism for attaching to a probe.

16. A method of forming a cryogenic probe of selected shape comprising
a) providing a plurality of removable cryogenic probe appliances of differing shapes, each of such cryogenic probe appliances comprising
(i) a proximal portion and a distal portion, the proximal portion being releasably attachable to a cryogenic probe;
(ii) the appliances further comprising a material having a high thermal conductivity for conducting heat between said proximal and distal portions;
b) selecting one of said appliances of the desired shape;
c) attaching the proximal portion of such selected appliance to a cryogenic probe.

17. The method of claim 16 wherein the material has a thermal conductivity of greater than about 100 W·m⁻¹·K¹.

18. The method of claim 16 wherein the material comprises aluminum.

19. The method of claim 16 wherein the material comprises copper.

20. The method of claim 16 wherein the material is malleable.

21. The method of claim 16 wherein at least the distal portion is flexible.

22. The method of claim 16 wherein the appliance is shaped to form a desired pattern of ablation in human tissue.

23. The method of claim 16 wherein the appliance is adapted to contact cardiac tissue.

24. The method of claim 16 wherein the appliance is shaped to form a line of ablation.

25. The method of claim 16 wherein the appliance is shaped to provide a two dimensional region of ablation.

26. The method of claim 16 wherein the appliance includes means for releasably attaching to a probe.

27. The method of claim 16 wherein the proximal portion includes threads for threadedly attaching the appliance to a probe.

28. The method of claim 16 wherein the appliance is adapted to frictionally engage a probe.

29. The method of claim 16 wherein the appliance includes a bayonet lock for attaching to a probe.

30. The method of claim 16 wherein the appliance includes a detent mechanism for attaching to a probe.

31. A method of providing a cryogenic probe appliance of selected shape comprising
a) providing a cryogenic probe appliance that is removably attachable to a cryogenic probe wherein at least a portion of the appliance is malleable to allow it to be formed into a desired shape;
b) bending said appliance to form a desired shape;
c) attaching the appliance to a cryogenic probe.

32. The method of claim 31 wherein the appliance includes means for releasably attaching to a probe.

33. The method of claim 31 wherein the appliance includes threads for threadedly attaching the appliance to a probe.

34. The method of claim 31 wherein the appliance is adapted to frictionally engage a probe.

35. The method of claim 31 wherein the appliance includes a bayonet lock for attaching to a probe.

36. The method of claim 31 wherein the appliance includes a detent mechanism for attaching to a probe.

37. The method of claim 31 wherein the appliance comprises a material having high thermal conductivity.

38. The method of claim 31 wherein the material comprises aluminum.

39. The method of claim 31 wherein the material comprises copper.

40. The method of claim 31 wherein the material is malleable.

41. The method of claim 31 wherein the material has a thermal conductivity of greater than about 100 W·m⁻¹·K⁻¹.

42. A method of using a cryogenic probe appliance comprising
a) providing a cryogenic probe appliance that is removably attachable to a cryogenic probe wherein at least a portion of the appliance is bendable to allow it to be formed into a desired shape;
b) attaching the appliance to a cryogenic probe;
c) bending the appliance to form a desired shape;
d) cooling the cryogenic probe and attached appliance;
e) contacting the appliance with human tissue to be treated.

43. The method of claim 42 wherein the appliance comprises a material having high thermal conductivity.

44. The method of claim 43 wherein the material comprises aluminum.

45. The method of claim 43 wherein the material comprises copper.

46. The method of claim 43 wherein the material is malleable.

47. The method of claim 42 wherein the appliance includes means for releasably attaching to a probe.

48. The method of claim 42 wherein the appliance includes threads for threadedly attaching the appliance to a probe.

49. The method of claim 42 wherein the appliance is adapted to frictionally engage a probe.

50. The method of claim 42 wherein the appliance includes a bayonet lock for attaching to a probe.

51. The method of claim 42 wherein the appliance includes a detent mechanism for attaching to a probe.

52. The method of claim 42 wherein the material has a thermal conductivity of greater than about 100 W·m⁻¹·K⁻¹

53. The method of claim 42 wherein the appliance is shaped to form a desired pattern of ablation in human tissue.

54. The method of claim 42 wherein the appliance is shaped to contact cardiac tissue.

55. The method of claim 54 wherein the appliance has a distal portion formed in a desired shape to contact cardiac tissue in a desired location.

56. The method of claim 42 wherein the appliance has a distal portion formed in a desired shape to contact cardiac tissue with sufficient precision so as not to damage surrounding tissue.

57. The method of claim 42 wherein the appliance has a distal portion formed in a desired shape to contact cardiac tissue with sufficient precision so as to prevent inadvertent ablation of surrounding tissue.

58. The method of claim 42 wherein the appliance has a distal portion formed in a desired shape to contact cardiac tissue in the proximity of the pulmonary vein.

59. The method of claim 42 wherein the appliance is shaped to form a line of ablation.

60. The method of claim 42 wherein the appliance is shaped to provide a two dimensional region of ablation.

61. The method of claim 42 wherein human tissue is contacted with the appliance for sufficient time to lower the temperature of the tissue.

62. The method of claim 42 wherein the appliance is shaped to form one or more lines of ablation in the Maze procedure.

63. A cryogenic probe system that includes
a cryogenic probe;
a removable cryogenic probe appliance for attachment to the cryogenic probe, the removable cryogenic probe appliance comprising a proximal portion and a distal portion, the proximal portion being releasably attachable to a cryogenic probe;
the appliance further comprising a material having high thermal conductivity for conducting heat between said proximal and distal portions.

64. The removable cryogenic probe appliance of claim 63 wherein at least the distal portion is flexible.

65. The removable cryogenic probe appliance of claim 63 wherein the material has a thermal conductivity of greater than about 100 W·m⁻¹·K⁻¹.

66. The removable cryogenic probe appliance of claim 63 wherein the material comprises aluminum.

67. The removable cryogenic probe appliance of claim 63 wherein the material comprises copper.

68. The removable cryogenic probe appliance of claim 63 wherein the material is malleable.

69. The removable cryogenic probe appliance of claim 63 wherein the appliance is shaped to form a desired pattern of ablation in human tissue.

70. The removable cryogenic probe appliance of claim 63 wherein the appliance is adapted to contact cardiac tissue.

71. The removable cryogenic probe appliance of claim 63 wherein the appliance is shaped to form a line of ablation.

72. The removable cryogenic probe appliance of claim 63 wherein the appliance is shaped to form a two dimensional region of ablation.

73. The removable cryogenic probe appliance of claim 63 wherein the proximal portion includes means for releasably attaching to a cryogenic probe.

74. The removable cryogenic probe appliance of claim 73 wherein the proximal portion of the appliance includes threads for releasable attachment to a cryogenic probe.

75. The removable cryogenic probe appliance of claim 73 wherein the appliance is frictionally engageable to a probe.

76. The removable cryogenic probe appliance of claim 73 wherein the proximal portion of the appliance includes a bayonet lock for attaching to a probe.

77. The removable cryogenic probe appliance of claim 73 wherein the proximal portion of the appliance includes a detent mechanism for attaching to a probe.
